(19) United States
(12) Patent Application Publication
(10) Pub. No.: US 2018/0345044 A1
(43) Pub. Date: Dec. 6, 2018

(54) ULTRASONIC MATERIAL, METHOD FOR PREPARING THE MATERIAL, AND ULTRASONIC PROBE COMPRISING THE MATERIAL

(71) Applicant: HUAZHONG UNIVERSITY OF SCIENCE AND TECHNOLOGY, Wuhan (CN)

(72) Inventors: Benpeng ZHU, Wuhan (CN); Mingke SHEN, Wuhan (CN); Xiaofei YANG, Wuhan (CN); Yue ZHANG, Wuhan (CN); Jun OUYANG, Wuhan (CN)

(21) Appl. No.: 15/712,099

(22) Filed: Sep. 21, 2017

(30) Foreign Application Priority Data
Jun. 6, 2017 (CN) 201710417275.9

(51) Int. Cl. A61N 7/00; B29C 64/314; B29C 64/135; A61B 8/00

(52) U.S. Cl. CPC A61N 7/00; A61B 8/42; B29C 64/135; B29C 64/314

(57) ABSTRACT

A method for preparing an ultrasonic material, including: 1) mixing methyl ethyl ketone with ethyl alcohol to prepare an azeotropic mixture; uniformly mixing carbon nanotube powders with a dispersant in the azeotropic mixture to yield a dispersoid; drying the dispersoid to yield dry carbon nanotube powders; 2) mixing the dry carbon nanotube powders in 1) with a light-cured resin to form a sizing mixture; 3) evenly distributing the sizing mixture in 2) over a plane of a mask image projection based stereo lithography apparatus to form a sizing mixture layer; 4) switching a design model of focused light-induced ultrasonic material to a two-dimensional image; projecting the two-dimensional image on a surface of the sizing mixture layer in 3); 5) exposing the sizing mixture layer in 3) under visible light and solidifying the sizing mixture layer; and 6) repeating 3)-5) to complete printing of the ultrasonic material.

### CROSS-REFERENCE TO RELATED APPLICATIONS

Pursuant to 35 U.S.C. § 119 and the Paris Convention Treaty, this application claims foreign priority to Chinese Patent Application No. 201710417275.9 filed Jun. 6, 2017, the contents of which are incorporated herein by reference. Inquiries from the public to applicants or assignees concerning this document or the related applications should be directed to: Matthias Scholl P.C., Attn.: Dr. Matthias Scholl Esq., 245 First Street, 18th Floor, and Cambridge, Mass. 02142.

### BACKGROUND OF THE INVENTION

### Field of the Invention

The invention relates to an ultrasonic material, a method for preparing the material, and an endoscopic photoinduced ultrasonic probe comprising the same.

### Description of the Related Art

Conventionally, the piezoelectric ultrasonic transducer is used to diagnose and treat blood vessel pathologies. However, low-frequency ultrasonic transducers are bulky, and inaccurate in their treatment effect.

In recent years, high intensity focused ultrasound (HIFU) technology has been developed and used for trauma therapy. The core technology of HIFU is focusing, and the common focusing mode is self-focusing; that is, the transducers are directly fabricated to possess a self-focusing curve. However, the piezoelectric material for manufacturing the transducers is high in hardness, poor in flexibility, which leads to a complex manufacturing process.

### SUMMARY OF THE INVENTION

In view of the above-described problems, it is one objective of the invention to provide an ultrasonic material, a method for preparing the same, and an endoscopic photoinduced ultrasonic probe comprising the same. The endoscopic photoinduced ultrasonic probe is small-sized and accurate in diagnosis and treatment, and meanwhile, the diagnosis and treatment can be performed simultaneously.

To achieve the above objective, in accordance with one embodiment of the invention, there is provided a method for preparing the ultrasonic material, comprising:
1) mixing methyl ethyl ketone with ethyl alcohol to prepare an azeotropic mixture; uniformly mixing carbon nanotube powders with a dispersant in the azeotropic mixture to yield a dispersoid; evaporating a solvent in the dispersoid, and drying the dispersoid for between 11 and 13 hrs at a temperature between 40 and 50° C. to yield dry carbon nanotube powders;
2) mixing the dry carbon nanotube powders in 1) with a light-cured resin to form a sizing mixture, a weight ratio of the dry carbon nanotube powders to the light-cured resin being 1: 80-99;
3) forming the focused light-induced ultrasonic material using a mask image projection based stereo lithography apparatus; moving a film collector to the left, and distributing the sizing mixture to a film collector to form a sizing mixture layer between 30 and 50 μm thick;
4) switching a computer aided design model to a two-dimensional image; projecting the two-dimensional image to a bottom of the film collector via a digital micromirror device on a platform;
5) reflecting visible light generated from LED light to the bottom of the film collector via the digital micromirror device on the platform; forming crosslinked matrix between networks of the sizing mixture layer using the light-cured resin in 2) by photopolymerization; moving the platform upwards when one layer of focused light-induced ultrasonic material is solidified; and
6) repeating 2)-5) to complete printing of the focused light-induced ultrasonic material according to the computer aided design model.

In a class of this embodiment, the carbon nanotube powders and the dispersant in the azeotropic mixture are ground using stainless steel grinding balls of a planetary ball mill for between 10 and 13 hrs at a speed between 150 and 250 rpm to yield the dispersoid. The dispersoid is dried for between 11 and 13 hrs at a temperature between 40 and 50° C. The dispersant is polyvinyl alcohol.

In a class of this embodiment, in 2), the dry carbon nanotube powders obtained in 1) are mixed with the light-cured resin by ball milling for between 1 and 2 hr(s) to yield the sizing mixture. A weight ratio of the dry carbon nanotube powders to the light-cured resin is 1: 80-99.

In a class of this embodiment, the carbon nanotube powders and the dispersant in the azeotropic mixture are ground using stainless steel grinding balls of a planetary ball mill for 12 hrs at a speed of 200 rpm to yield the dispersoid. The dispersoid is dried for 12 hrs at 50° C.

In a class of this embodiment, in 2), the dry carbon nanotube powders obtained in 1) are mixed with the light-cured resin by ball milling for 1 hr to yield the sizing mixture. A weight ratio of the dry carbon nanotube powders to the light-cured resin is 1:

99.

In a class of this embodiment, the sizing mixture in 2) is formed at a temperature of below 15° C. in vacuum. The carbon nanotube powders are mixed with the light-cured resin using the planetary ball mill for 1 hr to yield the sizing mixture

In accordance with another embodiment of the invention, there is provided a focused light-induced ultrasonic material, being prepared using the method for preparing the focused light-induced ultrasonic material.

In accordance with another embodiment of the invention, there is provided an endoscopic photoinduced ultrasonic probe, comprising a shell, a first incident optical fiber, a focused light-induced ultrasonic material, a total reflector, a cylindrical photoinduced ultrasonic material, and a second incident optical fiber. The first incident optical fiber is used for treatment, and the second incident optical fiber is used for imaging. The first incident optical fiber and the second incident optical fiber are parallel, and attached to each other. The first incident optical fiber and the second incident optical fiber each are sheathed in the shell. An end of the first incident optical fiber is connected to the focused light-induced ultrasonic material, and an end of the second optical fiber is connected to the cylindrical photoinduced ultrasonic material. A diameter of the first incident optical fiber equals to a diameter of the second incident optical fiber. The diameter of the second incident optical fiber is smaller than a diameter of the cylindrical photoinduced ultrasonic material. The diameter of the first incident optical fiber is smaller than a diameter of the focused light-induced ultrasonic material. A center of the second incident optical fiber, a center of the cylindrical photoinduced ultrasonic material, and an axis of the total reflector are on a same line. A center of the first incident optical fiber and a center of the focused light-induced ultrasonic material are on a same line. The cylindrical photoinduced ultrasonic material and the total reflector are contactless. The focused light-induced ultrasonic material is a concave spherical structure, and an angle between a cross section of the focused light-induced ultrasonic material and a horizontal line is between 45° and 60°. A focal point of the focused light-induced ultrasonic material and a reflected light ray of the total reflector always focus at a focus area. A normal of the cross section of the focused light-induced ultrasonic material and a normal of the total reflector are in a same plane. An angle between a mirror surface of the total reflector and a horizontal plane is 45°.

In a class of this embodiment, the first incident optical fiber and the second incident optical fiber are glass or plastic.

In a class of this embodiment, a distance from the center of the cylindrical photoinduced ultrasonic material to the axis of the total reflector is less than 1 mm.

In a class of this embodiment, a diameter of the cylindrical photoinduced ultrasonic material is between 2 and 3 mm A diameter of the focused light-induced ultrasonic material is between 2 and 5 mm.

Existing methods for preparing the focused light-induced ultrasonic material, including the manual semi-automatic method, are top-down processing methods which involve in polishing and press forming the bulk materials. The piezoelectric ceramics features high hardness and poor flexibility, thus is complex to process using the existing methods. Compared with the existing method for preparing the focused light-induced ultrasonic material, a method for preparing the focused light-induced ultrasonic material using the 3D printing is inventively put forward according to the concept of 3D printing in the invention. A mixture of carbon nanotube powders and light-cured resin is used as raw material, and is printed layer by layer from bottom to top by photocuring. The method is easy to operate, and many researches are focused on the method. Obviously, compared with the manual semi-automatic method, the method which is capable of printing out the focused light-induced ultrasonic material by setting parameters on the computer can accurately control the curvature and smoothness of the spherical surface, reduce error and energy loss, and more importantly, lay a foundation for the miniaturization of the focused light-induced ultrasonic material. Eventually, the high-intensity focused light-induced ultrasonic material which features accurate and controllable focal range and causes less energy lose is prepared using the method in the invention.

Advantages of the endoscopic photoinduced ultrasonic probe according to embodiments of the invention are summarized as follows:

1. Compared with the piezoelectric ultrasonic transducer, the endoscopic photoinduced ultrasonic probe in the embodiments of the invention uses dual optical fiber structure. The endoscopic photoinduced ultrasonic probe is sent to the blood vessel via a minimally invasive surgery. The second incident optical fiber and the cylindrical photoinduced ultrasonic probe work to find the pathological tissues, meanwhile the first incident optical fiber and the focused light-induced ultrasonic material work to smash the pathological tissues, causing less pain to the patient, shortening the treatment time, and improving the therapeutic efficiency.

2. Compared with the piezoelectric ultrasonic transducer, the endoscopic photoinduced ultrasonic probe in the embodiments of the invention is small in size, and can be accommodated in most of the blood capillaries. Therefore, the endoscopic photoinduced ultrasonic probe is capable of examining blind areas of conventional device, and a comprehensive examination of the blood vessel is performed using the endoscopic photoinduced ultrasonic probe. Therefore, pathological tissues in the early stage which hides in the small blood vessels can be timely detected and treated.

3. Compared with the piezoelectric ultrasonic transducer, the endoscopic photoinduced ultrasonic probe in the embodiments of the invention uses novel photoinduced ultrasonic material to diagnose and treat pathological tissues simultaneously. The center frequency of the cylindrical photoinduced ultrasonic material in the embodiments of the invention is much higher than conventional piezoelectric materials, therefore, the imaging quality of the cylindrical photoinduced ultrasonic material is much better than the imaging quality of conventional piezoelectric materials. The acoustic pressure of the focused light-induced ultrasonic material is relatively high, and the acoustic pressure output is appropriate, thus the treatment using the focused light-induced ultrasonic material is effective.

4. Compared with the piezoelectric ultrasonic transducer, the photoinduced ultrasonic conversion efficiency of the endoscopic photoinduced ultrasonic probe in the embodiments of the invention is high, thus a relatively weak light source can be converted to ultrasonic signals with large amplitude which leads to better imaging performance and effective treatment.

Advantages of the focused light-induced ultrasonic material, and the method for preparing the focused light-induced ultrasonic material according to embodiments of the invention are summarized as follows:

1. The method uses mask image projection based stereo lithography technology and 3D printing technology to accurately fabricate the focused light-induced ultrasonic material for the endoscopic photoinduced ultrasonic probe. The focused light-induced ultrasonic material prepared using the method features uniform structure, smooth appearance, accurate curvature of the spherical surface, good focusing effect, and high sensibility.

2. The focused light-induced ultrasonic material prepared using the method is elaborate, and is exactly the same as the computer-aided design model, thus the method reduces waste and error, increases the utilization ratio of the materials, improves the energy conversion efficiency of the focused light-induced ultrasonic material, and prolongs the service life.

### BRIEF DESCRIPTION OF THE DRAWINGS

The invention is described hereinbelow with reference to the accompanying drawings, in which:

FIG. 1 is a three-dimensional schematic diagram of an endoscopic photoinduced ultrasonic probe in accordance with one embodiment of the invention;

FIG. 2 is a cross-sectional view of an endoscopic photoinduced ultrasonic probe in accordance with one embodiment of the invention;

FIG. 3 is a three-dimensional schematic diagram of a focused light-induced ultrasonic material in accordance with one embodiment of the invention;

FIG. 4 is a diagram showing a production process of a focused light-induced ultrasonic material in accordance with one embodiment of the invention; and

FIG. 5 is a schematic diagram of a mask image projection based stereo lithography apparatus in accordance with one embodiment of the invention.

In the drawings, the following reference numbers are used: 1. Shell; 2. First incident optical fiber; 3. Focused light-induced ultrasonic material; 4. Total reflector; 5. Cylindrical photoinduced ultrasonic material; 6. Second incident optical fiber; 7. Focus area; 8. Cross section of focused light-induced ultrasonic material; 9. Sizing mixture distributor; 10. Platform; 11. Film collector; 12. LED light; and 13. Digital micromirror device.

### DETAILED DESCRIPTION OF THE EMBODIMENTS

For further illustrating the invention, experiments detailing a focused light-induced ultrasonic material, a method for preparing the same, and an endoscopic photoinduced ultrasonic probe comprising the same are described below. It should be noted that the following examples are intended to describe and not to limit the invention. In addition, the technical features mentioned in each example can be combined as long as the features do not conflict with each other.

### Example 1

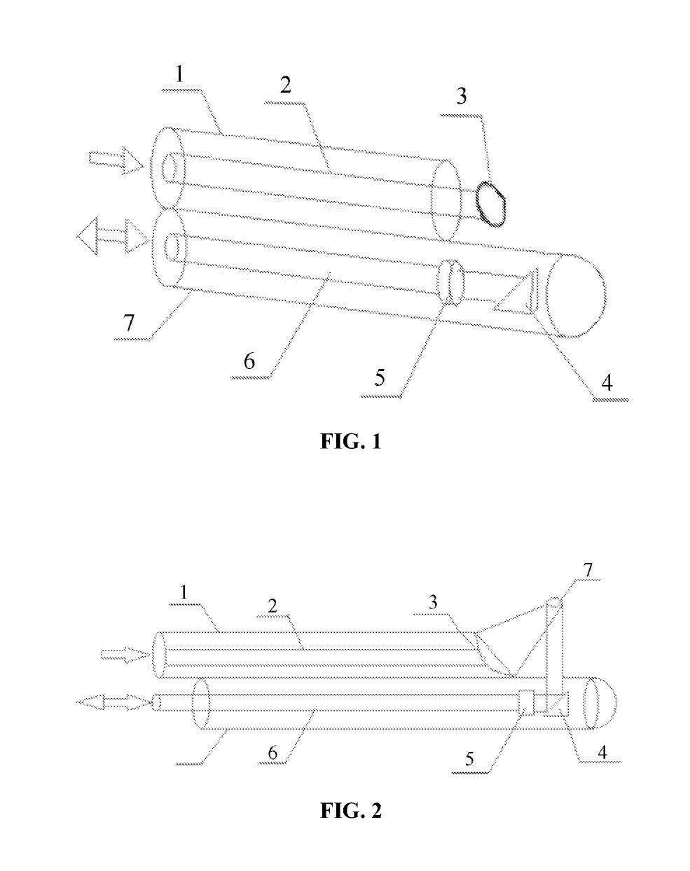

FIG. 1 is a three-dimensional schematic diagram of an endoscopic photoinduced ultrasonic probe. FIG. 2 is a cross-sectional view of the endoscopic photoinduced ultrasonic probe. A second incident optical fiber 6 is bonded with the cylindrical photoinduced ultrasonic material 5. A diameter of the cylindrical photoinduced ultrasonic material 5 is 2 mm A center of the second incident optical fiber 6 and a center of the cylindrical photoinduced ultrasonic material 5 are on the same line. A first incident optical fiber 2 is bonded with the focused light-induced ultrasonic material 3. A diameter of the focused light-induced ultrasonic material 3 is 2 mm. The first incident optical fiber is seamlessly connected to the focused light-induced ultrasonic material. A total reflector 4 is fixed at one side of the cylindrical photoinduced ultrasonic material 5 and is 1 mm away from the cylindrical photoinduced ultrasonic material. An axis of the total reflector 4 and a center of the cylindrical photoinduced ultrasonic material 5 are on the same line. The two incident optical fibers are sheathed in two shells 1. The shells are closely attached to each other, and are in parallel. An angle between a cross section 8 of the focused light-induced ultrasonic material 3 and the horizontal plane is 45°, and a normal of the cross section 8 of the focused light-induced ultrasonic material 3 and a normal of a mirror surface of the total reflector 4 are in the same plane. A focal point of the focused light-induced ultrasonic material 3 and a reflected light ray of the total reflector 4 always focus at a focus area 7. The endoscopic photoinduced ultrasonic probe is able to rotate 360 degrees.

The endoscopic photoinduced ultrasonic probe comprises the second incident optical fiber and the cylindrical photoinduced ultrasonic material. Pulsed light is emitted to the cylindrical photoinduced ultrasonic material 5 through the second incident optical fiber 6, and the cylindrical photoinduced ultrasonic material 5 vibrates to produce ultrasonic signals. Ultrasonic echo signals are generated when ultrasonic signals come across pathological tissues. Then a beam of continuous light is emitted along the same path to the cylindrical photoinduced ultrasonic material 5, and the cylindrical photoinduced ultrasonic material 5 shoots the beam out along the original path. The ultrasonic echo signals cause the cylindrical photoinduced ultrasonic material 5 to vibrate and change the light intensity of the continuous light. A photodetector works to receive and analyze the light intensity change of reflected continuous light so as to locate the pathology tissues. Another beam of pulsed light is emitted through the first incident optical fiber 2 to the focused light-induced ultrasonic material 3. As the light is focused, ultrasonic energy is enhanced and strong enough to smash the pathological tissues.

### Example 2

FIG. 1 is a three-dimensional schematic diagram of an endoscopic photoinduced ultrasonic probe. FIG. 2 is a cross-sectional view of the endoscopic photoinduced ultrasonic probe. A second incident optical fiber 6 is bonded with the cylindrical photoinduced ultrasonic material 5. A diameter of the cylindrical photoinduced ultrasonic material 5 is 3 mm A center of the second incident optical fiber 6 and a center of the cylindrical photoinduced ultrasonic material 5 are on the same line. A first incident optical fiber 2 is bonded with the focused light-induced ultrasonic material 3. A diameter of the focused light-induced ultrasonic material 3 is 5 mm. The first incident optical fiber is seamlessly connected to the focused light-induced ultrasonic material. A total reflector 4 is fixed at one side of the cylindrical photoinduced ultrasonic material 5 and is 1 mm away from the cylindrical photoinduced ultrasonic material. An axis of the total reflector 4 and a center of the cylindrical photoinduced ultrasonic material 5 are on the same line. The two incident optical fibers are sheathed in two shells 1, respectively. The shells are closely attached to each other, and are in parallel. An angle between a cross section 8 of the focused light-induced ultrasonic material 3 and the horizontal plane is 60°, and a normal of the cross section 8 of the focused light-induced ultrasonic material 3 and a normal of a mirror surface of the total reflector 4 are in the same plane. A focal point of the focused light-induced ultrasonic material 3 and a reflected light ray of the total reflector 4 always focus at a focus area 7. The endoscopic photoinduced ultrasonic probe is able to rotate 360 degrees.

The endoscopic photoinduced ultrasonic probe comprises the second incident optical fiber and the cylindrical photoinduced ultrasonic material. Pulsed light is emitted to the cylindrical photoinduced ultrasonic material 5 through the second incident optical fiber 6, and the cylindrical photoinduced ultrasonic material 5 vibrates to produce ultrasonic signals. Ultrasonic echo signals are generated when ultrasonic signals come across pathological tissues. Then a beam of continuous light is emitted along the same path to the cylindrical photoinduced ultrasonic material 5, and the cylindrical photoinduced ultrasonic material 5 shoots the beam out along the original path. The ultrasonic echo signals cause the cylindrical photoinduced ultrasonic material 5 to vibrate and change the light intensity of the continuous light. A photodetector works to receive and analyze the light intensity change of reflected continuous light so as to locate the pathology tissues. Another beam of pulsed light is emitted through the first incident optical fiber 2 to the focused light-induced ultrasonic material 3. As the light is focused, ultrasonic energy is enhanced and strong enough to smash the pathological tissues.

### Example 3

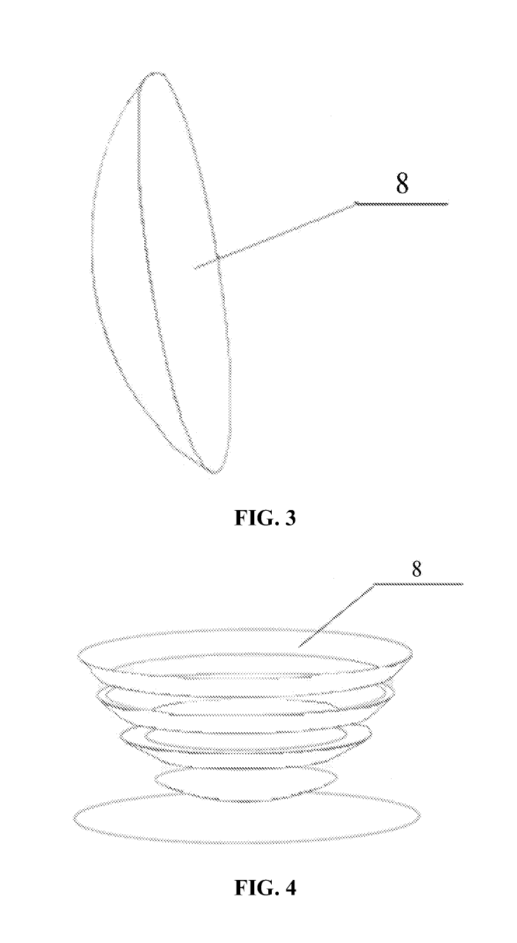

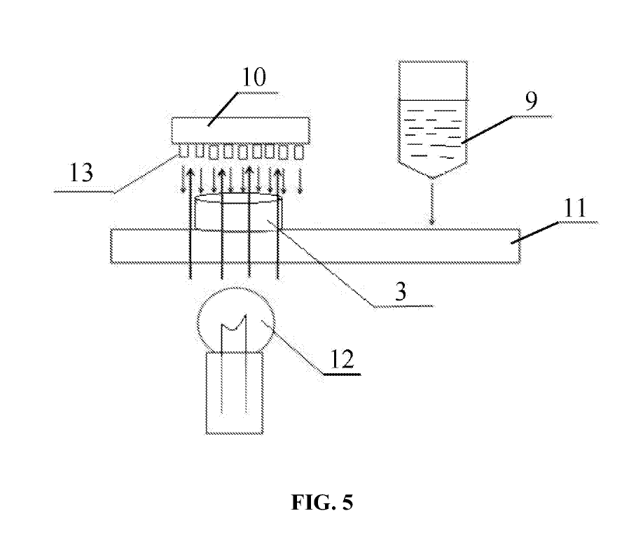

FIG. 3 is a three-dimensional schematic diagram of a focused light-induced ultrasonic material 3, and FIG. 4 is a diagram showing a production process of the focused light-induced ultrasonic material 3. FIG. 5 is a schematic diagram of a mask image projection based stereo lithography apparatus. The focused light-induced ultrasonic material 3 is prepared by two steps: 1) methyl ethyl ketone was mixed with ethyl alcohol to form azeotropic mixture. Carbon nanotube powders and dispersant in the azeotropic mixture were ground using stainless steel grinding balls of a planetary ball mill for 10 hrs at a speed of 150 rpm to yield a dispersoid. The dispersoid was dried for 11 hrs at 40° C. Dry carbon nanotube powders were yielded when solvent in the dispersoid was evaporated. 2) The dry carbon nanotube powders in 1) were mixed with the light-cured resin SI500 by ball milling for 1 hr to form a sizing mixture, and a weight ratio of the dry carbon nanotube powders to the light-cured resin SI500 was 1:80. The mask image projection based stereo lithography technology and 3D printing technology were used, and a stereo lithography apparatus was employed. When a film collector 11 was moved to the left, a sizing mixture distributor worked to distribute the sizing mixture to the film collector 11, and a thin layer was formed by using a scraper. A computer aided design model was switched to a two-dimensional image, and the two-dimensional image was projected to a bottom of the film collector 11 via a digital micromirror device 13. Visible light generated by an LED light 12 was reflected to the bottom of the film collector 11 via the digital micromirror device on the platform 10. Light-cured resin in the sizing mixture is photopolymerized and formed crosslinked matrix between networks of the polymers, and the platform 10 was moved upwards when one layer of focused light-induced ultrasonic material was solidified. More sizing mixture was added in the sizing mixture distributor 9 and was distributed to the film collector 11 to repeat the above steps until the printing of the focused light-induced ultrasonic material was completed.

### Example 4

FIG. 3 is a three-dimensional schematic diagram of a focused light-induced ultrasonic material 3, and FIG. 4 is a diagram showing a production process of the focused light-induced ultrasonic material 3. FIG. 5 is a schematic diagram of a mask image projection based stereo lithography apparatus. The focused light-induced ultrasonic material 3 is prepared by two steps: 1) methyl ethyl ketone was mixed with ethyl alcohol to form azeotropic mixture. Carbon nanotube powders and dispersant in the azeotropic mixture were ground using stainless steel grinding balls of a planetary ball mill for 13 hrs at a speed of 250 rpm to yield a dispersoid. The dispersoid was dried for 13 hrs at 50° C. Dry carbon nanotube powders were yielded when solvent in the dispersoid was evaporated. 2) The dry carbon nanotube powders in 1) were mixed with the light-cured resin SI500 by ball milling for 2 hrs to form a sizing mixture, and a weight ratio of the dry carbon nanotube powders to the light-cured resin SI500 was 1:99. The mask image projection based stereo lithography technology and 3D printing technology were used, and a stereo lithography apparatus was employed. When a film collector 11 was moved to the left, a sizing mixture distributor 9 worked to distribute the sizing mixture to the film collector 11, and a thin layer was formed by using a scraper. A thickness thereof is 30 μm. A computer aided design model was switched to a two-dimensional image, and the two-dimensional image was projected to a bottom of the film collector 11 via a digital micromirror device 13. Visible light generated by an LED light 12 was reflected to the bottom of the film collector 11 via the digital micromirror device on the platform 10. Light-cured resin in the sizing mixture was photopolymerized and formed crosslinked matrix between networks of the polymers, and the platform 10 was moved upwards when one layer of focused light-induced ultrasonic material was solidified. More sizing mixture was added in the sizing mixture distributor 9 and was distributed to the film collector 11 to repeat the above steps until the printing of the focused light-induced ultrasonic material was completed.

### Example 5

FIG. 3 is a three-dimensional schematic diagram of a focused light-induced ultrasonic material 3, and FIG. 4 is a diagram showing a production process of the focused light-induced ultrasonic material 3. FIG. 5 is a schematic diagram of a mask image projection based stereo lithography apparatus. The focused light-induced ultrasonic material 3 is prepared by two steps: 1) methyl ethyl ketone was mixed with ethyl alcohol to form azeotropic mixture. Carbon nanotube powders and dispersant in the azeotropic mixture were ground using stainless steel grinding balls of a planetary ball mill for 12 hrs at a speed of 200 rpm to yield a dispersoid. The dispersoid was dried for 12 hrs at 50° C. Dry carbon nanotube powders were yielded when solvent in the dispersoid was evaporated. 2) The dry carbon nanotube powders in 1) were mixed with the light-cured resin SI500 by ball milling for 1 hr to form a sizing mixture, and a weight ratio of the dry carbon nanotube powders to the light-cured resin SI500 was 1:99. The mask image projection based stereo lithography technology and 3D printing technology were used, and a stereo lithography apparatus was employed. When a film collector 11 was moved to the left, a sizing mixture distributor 9 worked to distribute the sizing mixture to the film collector 11, and a thin layer was formed by using a scraper. A thickness thereof is 50 μm. A computer aided design model was switched to a two-dimensional image, and the two-dimensional image was projected to a bottom of the film collector 11 via a digital micromirror device 13. Visible light generated by an LED light 12 was reflected to the bottom of the film collector 11 via the digital micromirror device on the platform 10. Light-cured resin in the sizing mixture was photopolymerized and formed crosslinked matrix between networks of the polymers, and the platform 10 was moved upwards when one layer of focused light-induced ultrasonic material was solidified. More sizing mixture was added in the sizing mixture distributor 9 and was distributed to the film collector 11 to repeat the above steps until the printing of the focused light-induced ultrasonic material was completed.

Unless otherwise indicated, the numerical ranges involved in the invention include the end values. While particular embodiments of the invention have been shown and described, it will be obvious to those skilled in the art that changes and modifications may be made without departing from the invention in its broader aspects, and therefore, the aim in the appended claims is to cover all such changes and modifications as fall within the true spirit and scope of the invention.

The invention claimed is:

1. A method for preparing an ultrasonic material, the method comprising:
1) mixing methyl ethyl ketone with ethyl alcohol to prepare an azeotropic mixture; uniformly mixing carbon nanotube powders with a dispersant in the azeotropic mixture to yield a dispersoid; drying the dispersoid for between 11 and 13 hrs at a temperature between 40 and 50° C. to yield dry carbon nanotube powders;
2) mixing the dry carbon nanotube powders in 1) with a light-cured resin to form a sizing mixture, a weight ratio of the dry carbon nanotube powders to the light-cured resin being 1: 80-99;
3) evenly distributing the sizing mixture in 2) over a plane of a mask image projection based stereo lithography apparatus to form a sizing mixture layer between 30 and 50 μm thick;
4) switching a design model of an ultrasonic material to a two-dimensional image; projecting the two-dimensional image on a surface of the sizing mixture layer in 3);
5) exposing the sizing mixture layer in 3) under visible light, forming, by the light-cured resin in the sizing mixture, crosslinked matrix through photopolymerization, and solidifying the sizing mixture layer according to the two-dimensional image in 4); and
6) repeating 3)-5) to complete printing of the ultrasonic material according to the design model of the ultrasonic material.

2. The method of claim 1, wherein
in 1), the carbon nanotube powders and the dispersant in the azeotropic mixture are ground using stainless steel grinding balls of a planetary ball mill for between 10 and 13 hrs at a speed between 150 and 250 rpm to yield the dispersoid; the dispersoid is dried for between 11 and 13 hrs at a temperature between 40 and 50° C.; the dispersant is polyvinyl alcohol; and
in 2), the dry carbon nanotube powders obtained in 1) are mixed with the light-cured resin by ball milling for between 1 and 2 hr(s) to yield the sizing mixture; and a weight ratio of the dry carbon nanotube powders to the light-cured resin is 1: 80-99.

3. The method of claim 1, wherein
in 1), the carbon nanotube powders and the dispersant in the azeotropic mixture are ground using stainless steel grinding balls of a planetary ball mill for 12 hrs at a speed of 200 rpm to yield the dispersoid; the dispersoid is dried for 12 hrs at 50° C.; and
in 2), the dry carbon nanotube powders obtained in 1) are mixed with the light-cured resin by ball milling for 1 hr to yield the sizing mixture; and a weight ratio of the dry carbon nanotube powders to the light-cured resin is 1:99.

4. The method of claim 1, wherein in 2), the sizing mixture is formed at a temperature of below 15° C. in vacuum; and the carbon nanotube powders are mixed with the light-cured resin using the planetary ball mill for 1 hr to yield the sizing mixture.

5. An ultrasonic material prepared by the method of claim 1.

6. An ultrasonic probe, comprising:
a shell;
a first incident optical fiber;
an ultrasonic material;
a total reflector;
a cylindrical photoinduced ultrasonic material; and
a second incident optical fiber;
wherein
the first incident optical fiber is used for treatment, and the second incident optical fiber is used for imaging;
the first incident optical fiber and the second incident optical fiber are parallel, and attached to each other; the first incident optical fiber and the second incident optical fiber each are sheathed in the shell; an end of the first incident optical fiber is connected to the ultrasonic material, and an end of the second optical fiber is connected to the cylindrical photoinduced ultrasonic material; a diameter of the first incident optical fiber equals to a diameter of the second incident optical fiber; the diameter of the second incident optical fiber is smaller than a diameter of the cylindrical photoinduced ultrasonic material; the diameter of the first incident optical fiber is smaller than a diameter of the ultrasonic material; and
a center of the second incident optical fiber, a center of the cylindrical photoinduced ultrasonic material, and an axis of the total reflector are on a same line; a center of the first incident optical fiber and a center of the ultrasonic material are on a same line; the cylindrical photoinduced ultrasonic material and the total reflector are contactless; the ultrasonic material is a concave spherical structure, and an angle between a cross section of the ultrasonic material and a horizontal line is between 45° and 60°; a focal point of the ultrasonic material and a reflected light ray of the total reflector always focus at a focus area; a normal of the cross section of the ultrasonic material and a normal of the total reflector are in a same plane; and an angle between a mirror surface of the total reflector and a horizontal plane is 45°.

7. The probe of claim 6, wherein the first incident optical fiber and the second incident optical fiber are glass or plastic.

8. The probe of claim 6, wherein a distance from the center of the cylindrical photoinduced ultrasonic material to the axis of the total reflector is less than 1 mm.

9. The probe of claim 6, wherein a diameter of the cylindrical photoinduced ultrasonic material is between 2 and 3 mm; and a diameter of the ultrasonic material is between 2 and 5 mm.
